(19) United States
(12) Patent Application Publication
(10) Pub. No.: US 2012/0070819 A1
(43) Pub. Date: Mar. 22, 2012

(54) Determination of Interactions of Constant Parts of Antibodies with FC-Gamma Receptors

(76) Inventors: Hartmut Hengel, Dusseldorf (DE); Ulrich Kalinke, Hannover (DE)

(21) Appl. No.: 13/257,119

(22) PCT Filed: Mar. 17, 2010
(86) PCT No.: PCT/EP2010/001683

§ 371 Date: Nov. 28, 2011

(30) Foreign Application Priority Data
Mar. 17, 2009 (DE) 10 2009 013 748.3

(51) Int. Cl. G01N 33/577; C12N 5/10; C12Q 1/70; C12N 15/85

(52) U.S. Cl. 435/5; 435/320.1; 435/455; 435/325; 435/355; 435/372; 435/7.23

(57) ABSTRACT

The invention relates to a novel method for the exact determination of the binding of the Fc-part of IgG-antibodies to Fc-gamma receptors, and for the simultaneous examination of the antigen-specificity and the Fc-gamma-receptor activation, as well as specific materials for use in said method. The invention furthermore relates to a method for identifying substances that affect the binding of the Fc-part of IgG-antibodies to Fc-gamma receptors, on the basis of the method for the exact determination of the binding of the Fc-part.

The invention relates to a novel method for the exact determination of the binding of the Fc-part of IgG-antibodies to Fc-gamma receptors, and for the simultaneous examination of the antigen-specificity and the Fc-gamma-receptor activation, as well as specific materials for use in said method. The invention furthermore relates to a method for identifying substances that affect the binding of the Fc-part of IgG-antibodies to Fc-gamma receptors, on the basis of the method for the exact determination of the binding of the Fc-part.

### BACKGROUND OF THE INVENTION

The fragment-crystallizable region (Fc region) is the region of an antibody that interacts with cell surface-receptors (Fc receptors) and some of the proteins of the complement system. The domain C_{H}3 is the Fc-receptor-binding site for opsonization, which binds to the CR1-receptor on phagocytes (monocytes, macrophages, neutrophil granulocytes, and a fraction of the dendritic cells), and thereby, amongst others, initiates the phagocytosis of the labeled particle. Thus, the opsonization allows the antibodies to activate the immune system. In IgG, IgA, and IgD antibody-isotypes, the Fc region is composed of two identical protein fragments that are derived from the second and third constant domains of the heavy chain of the antibody.

All Fcγ-receptors belong to the immunoglobulin-superfamily and are the most important Fc receptors in the induction of the phagocytosis of opsonized microbes. The family includes several members, FcγRI (CD64), FcγRIIA (CD32), FcγRIIB (CD32), FcγRIIIA (CD16a), FcγRIIIB (CD16b), and FcRn.

The fields for a use of medicaments on the basis of monoclonal antibodies (mAbs) are wide-spread. Today, monoclonal antibodies are increasingly used in oncology, in infectious diseases, the musculoskeletal system, disorders of endocrine and metabolic functions, in hematology, in respiratory diseases, diseases of the CNS, and in immunology, and in inflammatory diseases.

In the development and testing of new mAbs, it is amongst others essential for the safety and efficiency of these reagents to ensure the integrity and functionality of the mAbs by a continuous control.

Currently, the integrity of the constant part of monoclonal antibodies (mAbs) can be determined via the complement-mediated lysis of erythrocytes. This test is very imprecise and does not provide information regarding the relevant in vivo binding of the Fc-receptors. Nevertheless, information with respect to such interactions is of great importance since depending from the kind of expression the constant parts of the mAbs of the same subclass can undergo different interactions with Fc-receptors.

Vrdoljak et al. (in Vrdoljak A, Trescec A, Benko B, Simic M. A microassay for measurement of Fc function of human immunoglobulin preparations by using tetanus toxoid as antigen. Biologicals. 2004 June; 32 (2):78-83) describe a modified assay for the Fc-function of immunoglobulins based on the European Pharmacopoeia (EP). Thereby, in the assay tetanus toxoid is used as an alternative target instead of Rubella antigen, and the method is adjusted to microtiter plates.

Perez-del-Pulgar (in: Perez-del-Pulgar S, Lopez M, Gensana M, Jorquera J I. Possible alternative to European Pharmacopoeia's method of analysis Test for Fc Function of Immunoglobulin (2.7.9) by using tetanus toxoid as antigen. Pharmeur Sci Notes. 2006 August; 2006 (1):23-6) also describe tetanus toxoid as an alternative target in the assay of the EP.

Reipert, B. M. et al (in: Fc function of a new intravenous immunoglobulin product: IGIV 10% triple virally inactivated solution. Vox Sanguinis: Volume 91 (3) October 2006 p 256-263) describe the use of the method of the EP in combination with a flow-cytometry binding assay for the evaluation of the Fc-function.

For the further development of mAbs as effective medicaments, a need exists for a precise and reliable method for determining the interactions of the Fc-part of an antibody with Fc-receptors. The test as performed in accordance with the EP despite modifications is imprecise and does not provide information about the relevant in vivo binding and activation of the Fc-receptors. It is therefore an object of the invention to provide an improved test for the exact determination of the binding of the Fc-part of IgG-antibodies to Fc-gamma-receptors and their subsequent activation, as well as the specific materials to be used in said method. Additional advantages and objects will become apparent from the following more detailed description.

In a first aspect of the invention, the object is solved by a method for measuring the strength of the interaction between the constant parts of a monoclonal antibody and an Fc-receptor, wherein said method comprises the steps of: a) contacting a recombinant mammalian lymphoma cell according to the present invention with the constant parts of a monoclonal antibody, and b) measuring the expression of IL-2 from the recombinant mammalian lymphoma cell, wherein the strength of the expression of IL-2 is a measure for the strength of the interaction.

Preferred is a method according to the present invention, wherein simultaneously with measuring the strength of the interaction with the Fc-gamma-receptor and the activation, an examination of the antigen specificity of the monoclonal antibody as examined can take place. For this, the antibodies can be brought in contact with the antigens to be examined in a test system (in solution or bound to a surface, e.g. to a membrane, but also a cell, preferred examples are ELISA or “surface plasmon resonance” technology), and then a measuring of the strength of the interaction between the constant parts of the monoclonal antibody and an Fc-receptor according to the invention is performed. How to perform respective tests, also in a “high-throughput-approach”, is known to the person of skill from the literature. For a testing, the antibodies can be present in different forms, such as, for example, soluble, bound to a surface (e.g. plastics), in immune complexes or bound to target cells.

In the context of the present invention in a preferred case transfectants of a mouse-thymoma cell line were generated, which express the fusion molecules of the extracellular parts of the human Fc-receptors CD16, CD32, CD64, and the transmembrane region of the zeta-chain and the intracellular signaling domain of the T-cell receptor zeta-chain auf their surface. With this, the examination of interactions with the constant parts of mAbs becomes possible.

Thereby, an additional preferred aspect of the invention is a recombinant expression vector, comprising sequences for a recombinant expression of at least one fusion protein on the surface of a mammalian cell, wherein the recombinant expression vector comprises a) an extracellular part of a mammalian-Fc-receptor, b) the transmembrane region of the zeta-chain and c) the intracellular signaling domain of the T-cell receptor zeta-chain. Respective suitable expression vectors are known to the person of skill and contain sequences that are suitable for the expression of the desired fusion proteins in the respective host cell and the replication/selection, such as, for example, promoters, e.g. CMV promoter, T7 promoter, SV40 promoter, bla promoter, multiple cloning site(s), polyadenylation sequences, f1 origin, pUC origin, neomycin resistance gene; ampicillin resistance gene, and ribosome binding site(s).

Preferred is a recombinant expression vector of the invention, wherein the mammalian cell is selected from a zeta-chain-deficient cell, in particular a human or mouse-lymphoma cell, such as, for example, BW 5147 (ATCC TIB 47).

Preferred is a recombinant expression vector of the invention, wherein the receptor is selected from Fc-gamma-receptors, such as, for example, FcγRI (CD64), FcγRIIA (CD32), FcγRIIB (CD32), FcγRIIIA (CD16/CD16a), FcγRIIIB (CD16/CD16b) and FcRn, in particular selected from CD16, CD32 and CD64. In a preferred variant of the recombinant expression vector of the invention, the Fc-receptor-zeta-chain is fused with Fc-receptors for other Ig-subclasses, such as, for example, FcER. By producing and the stable expression of Fc-receptor-zeta-chain-fusion proteins of other subclasses, it becomes possible to also detect IgE-antibodies, e.g. in allergy diagnostics (regarding this, see also below). Furthermore, the extracellular domains of Fcγ-receptors of other species that are relevant for preclinical tests (for example non-human primates such as Makaka mulatta or Makaka fascicularis) can be expressed.

An additional preferred aspect of the invention then relates to a method for producing a recombinant mammalian lymphoma cell, comprising transfection of a mammalian lymphoma cell with an expression vector of the invention as above, and expression of at least one fusions proteins on the surface of the host cell. Preferably, the cell is selected from a human or mouse-lymphoma cell line, such as, for example, BW 5147 (ATCC TIB 47).

Yet another preferred aspect of the invention then relates to the recombinant mammalian lymphoma cell, produced according to a method as above. The cell expresses at least one fusion protein of the invention, nevertheless, can also express several proteins (e.g. from several expression vectors), in order to produce a “portfolio” of Fc-gamma-receptors on the surface. Preferred examples are CD16 and CD32; CD16 and CD64; CD16 and FcRn; CD32 and FcRn; CD32 and CD64; CD64 and FcRn. Preferably, the cell is selected from a human or mouse-lymphoma cell line, such as, for example, BW 5147 (ATCC TIB 47).

The invention relates to a method for the exact determination of the binding of the Fc-part of IgG-antibodies to Fc-gamma-receptors, and furthermore to a method for the simultaneous testing of the antigen specificity and the Fc-gamma-receptor activation. For this, transfectants of a mouse-thymoma cell line were generated by the inventors that expressed the fusion molecules of the extracellular parts of the human Fc-receptors CD16, CD32, CD64 and the transmembrane region and the intracellular signaling domain of the T-cell receptor zeta-chain on their surface. Using these, the interaction with the constant parts of mAbs is studied. In doing so, these can be present in different forms, such as, preferably soluble, further preferred bound to plastics, in immune complexes or bound to target cells.

For a detection of the interaction, use is made of the fact that the cytokine IL-2 is expressed depending from the strength of the binding of an mAb to a Fc-receptor. Based on the IL-2 level, therefore the strength of the interaction between a selected mAb and the different Fc-receptors can be determined precisely.

Furthermore, the method according to the invention allows for a measuring of the direct binding of labeled mAbs to the transfectants. Respective labels are known to the person of skill, such as, for example, radioactive or fluorescent labels.

Particular advantages of the invention, amongst others, are based on the fact that the binding behavior of soluble mAbs, of mAbs in immune complexes, and of mAbs that are bound to target cells can be studied. Furthermore, the interactions of each Fc-receptor can be studied individually. In addition, the invention allows for the determination of a precise binding profile of the mAbs of different subclasses to the individual Fc-receptors that are expressed in the mammal, and preferably the human. Thus, for the first time, conclusions are possible about the in vivo relevant binding and activation of the Fc-receptors.

The present invention can be used as a test during the development and Erprobung of new mAbs. In addition, on the basis of the present invention it is possible to provide new analogous testing methods for the detection of Fe-gamma-receptor activating autoimmune antibodies.

Further preferred is a method according to the invention as above, which furthermore comprises the generation of a binding profile of the constant part of mAbs of different subclasses for the individual Fc-receptors as expressed in a mammal.

An additional aspect of the present invention then relates to a method for identifying of compounds that influence the interaction between the constant parts of a monoclonal antibody and an Fc-receptor, comprising a) contacting of a recombinant mammalian lymphoma cell of the invention with the constant parts of a monoclonal antibody in the presence of a candidate compound, b) measuring the expression of IL-2 of the rekombinant mammalian lymphoma cell, wherein the strength of the expression of IL-2 is a measure for the strength of the interaction, and c) comparing the expression as measured in step b) with the IL-2 expression in the absence of the candidate compound.

The potential candidate compound, whose effect on the interaction between den constant parts of a monoclonal antibody and an Fc-receptor shall be identified, can be any chemical substance or a mixture thereof. For example, it can be a substance of a peptide library, a combinatory library, a cell extract, a “small molecular drug”, a protein, and/or a protein fragment.

In the present invention, the term “contacting” shall mean any interaction between the potentially interacting substance(s) with the constant part of a monoclonal antibody or the Fc-receptor, wherein each of the two components can be independently present in a liquid phase, for example in solution or in suspension, or bound to a solid phase, for example in form of an essentially plane surface or in form of particles, beads, or the like. In a preferred embodiment a multitude of different potential binding candidate compounds is immobilized on a solid surface, such as, for example on a compound library-chip, and the fusion proteins/antibodies of the present invention are subsequently brought in contact with such a chip.

Preferred is a method according to the invention for identifying of compounds that influence the interaction between the constant parts of a monoclonal antibody and an Fc-receptor, wherein the constant parts are present in the form of soluble mAbs, in mAbs bound to plastics, in immune complexes or bound to target cells. The above method preferably can be partially or fully performed in vitro in a recombinant cell, such as, for example, the one according to the invention as described above.

Further preferred is a method according to the invention for identifying of compounds that influence the interaction between the constant parts of a monoclonal antibody and an Fc-receptor, wherein the constant parts are labeled and/or are present in labeled mAbs. A measuring of the interaction can be achieved by a measuring of a label, which is either attached to the proteins and/or the potentially interacting compound. Suitable labels are known to the person of skill, and, for example, include fluorescent or radioactive labels. The binding of the components can also be detected through the change of electrochemical parameters of the interacting compound or the proteins, e.g. a change of the redox characteristics of either the protein/the proteins or of the interacting compound after binding. Suitable methods for a detection of such changes include, for example, potentiometric methods. Additional methods for the detection and/or measuring the binding of the components with each other are known in the state of the art.

Further preferred is a method according to the invention, furthermore comprising the simultaneous generation of a binding profile of the constant part of mAbs of different subclasses for the individual Fc-receptors as expressed in the mammal.

Further preferred is a method according to the invention, wherein the Method furthermore comprises the modification of the constant part and/or the candidate-compound for an increase or decrease of the strength of the binding to an Fc-receptor.

If, when using the method according to the invention, a compound can be found that influences the interaction between the constant parts of a monoclonal antibody and an Fc-receptor, according to the invention this compound becomes a “lead-compound” for the further commercial development of a medicament. Amongst others, this compound is used in the following, in particular living, test systems, and developed further.

A further preferred embodiment of the method of the present invention thus comprises the step of a chemical derivatization of the compound(s) as selected above. As used herein, in the context of the present invention a “derivative” shall mean a compound which is derived from a compound as identified according to the present invention, which, for example, is substituted with different moieties, and mixtures of different of these compounds which, for example, when adjusted to the actual disease to be treated, and/or to the patient on the basis of diagnostic data or data regarding the success or progression of the treatment, can be processed into a “personalized” medicament. In the context of the present invention, a “chemical derivatization” shall mean the process for a corresponding chemical modification, such as, for example, the substitution of different moieties. Preferably, a chemical derivatization is performed in order to affect an improved bioavailability, or a reduction of possible side-effects. In the context of the present invention, a “derivative” shall also mean a “precursor” of a substance, which during its administration for a treatment is modified in such a way because of the conditions in the body (e.g. pH in the stomach, or the like), or is metabolized by the body after uptake in such a way that the compound according to the invention or derivatives thereof is formed as an active substance.

A further aspect of the present invention then relates to a method for producing a pharmaceutical composition, comprising a) identifying of a compound that influences the interaction between the constant parts of a monoclonal antibody and an Fc-receptor using a method according to the invention as above, and b) mixing of the compound with a suitable pharmaceutical carrier and/or other suitable pharmaceutical auxiliary agents and additives, for example a suitable pharmaceutical carrier.

The production of pharmaceutical compositions, e.g. in form of medicaments including an amount of a compound according to the invention or their use according to the invention takes place in a common manner following commonly known methods of pharmaceutical technology. For this, the compounds are processed together with suitable pharmaceutically acceptable auxiliary agents and carriers into medication forms that are suitable for the different indications and application sites.

In doing so, the medicaments can be produced in a way that the respective desired release rate, e.g. a quick accumulation and/or a retardation or depot effect, respectively, are achieved. Thereby, a medicament can be an ointment, gel, patch, emulsion, lotion, foam, crème or mixed-phase or amphiphilic emulsion systems (oil/water-water/oil-mixed phase), liposome, transfersome, paste or powder.

According to the invention, the term “auxiliary agent” means any non-toxic, solid or liquid filling, diluting or packing material, as long as it does not react excessively disadvantageous with a compound or the patient. Liquid galenical auxiliary agents, for example, are sterile water, physiological saline, sugar solutions, ethanol and/or oils. Galenical auxiliary agents for the production of tablets and capsules can be, for example, binders and fillers.

Furthermore, a compound according to the invention can be used in the form of systemically applied medicaments. These include the parenteralia, which include the injectables and infusions. Injectables are either prepared in the form of ampoules or as so-called ready-for-use injectables, e.g. as ready-to-use injections or one-way injections, and also in vials with a rubber stopper for repeated withdrawals. The administration of the injectables can be carried out in the form of the subcutaneous (s.c.), intramuscular (i.m.), intravenous (i.v.) or intracutaneous application. In particular, the respective suitable form for injection can be produced as suspensions of crystals, solutions, nanoparticular or colloidal-disperse systems, such as, for example hydrosols.

Furthermore, the injectable preparations can be produced as concentrates that are dissolved or dispersed with aqueous isotonic diluents. The infusions can also be prepared in form of isotonic solutions, fatty emulsions, liposome preparations, or micro-emulsions. Similar to the injectables, the preparations for infusion can also be prepared in the form of concentrates for a dilution. The injectable preparations can also be administered in the form of continuous infusions, both in the stationary and in ambulant therapy, e.g. in the form of mini-pumps.

The compound according to the invention in the parenteralia can be bound to micro-carriers or nanoparticles, for example to micro-disperse particles on the basis of poly(meth)acrylates, polylactates, polyglycolates, polyamine acids, or polyether urethanes. The parenteral preparations can also be modified as depot preparations, e.g. based on the “multiple unit principle”, when an inhibitor according to the invention is included in micro-disperse or dispersed, suspended form or as crystal suspension, respectively, or based on the “single unit principle”, when an inhibitor according to the invention is included in a dosage form, e.g. a tablet or a rod, which is subsequently implanted. Often, the implants or depot dosage form in the case of “single unit” and “multiple unit” dosage forms consist of so-called biodegradable polymers, such as, for example, polyesters of lactic and glycolic acid, polyetherurethanes, polyamino acids, poly(meth)acrylates or polysaccharides.

As auxiliary agents and carriers for the production of parenteralia aqua sterilisata, substances influencing the pH, such as, for example, organic and inorganic acids and bases as well as their salts, buffers for adjusting the pH, isotonic agents, such as, for example, sodium chloride, sodium hydrogen carbonate, glucose and fructose, tensides or surfactants, respectively, and emulgators, such as, for example, partial fatty acid esters of polyoxyethylene sorbitane (Tween®) or e.f. fatty acid esters of polyoxyethylene (Cremophor®), fatty oils, such as, for example, peanut oil, soy bean oil and castor oil, synthetic fatty acid esters, such as, for example, ethyl oleate, isopropyl myristate and neutral oil (Miglyol®), as well as polymeric excipients, such as, for example, gelatin, dextran, polyvinyl pyrrolidone, additives increasing the solubility, organic solvents, such as, for example, propylene glycol, ethanol, N,N-dimethyl acetamide, propylene glycol, or complex-forming agents, such as, for example, citrates and urea, preservatives, such as, for example, benzoic acid hydroxypropyl ester and -methyl ester, benzyl alcohol, antioxidants, such as, for example, sodium sulfite and stabilizators, such as, for example, EDTA, can be used.

Thickening agents are added in suspensions, in order to avoid the sedimentation of the inhibitor according to the invention of tensides and peptide stabilisators, in order to ensure the dispersability of the sediment, or of complexing agents, such as EDTA. Drug complexes can also be obtained using different polymers, for example with polyethylene glycoles, polystyrenes, carboxy methylcellulose, Pluronics®, or polyethylene glycole sorbite fatty acid esters. For the production of lyophilisates, scaffolding agents are used, such as, for example, mannitol, dextran, sucrose, human albumin, lactose, PVP or gelatins.

The actual suitable dosage forms can be produced in agreement with recipes and methods that are known to the person of skill and based pharmaceutical-physical principles.

In an additional embodiment of the present invention the medicament that is used according to the present invention can be administered via different routes, for example orally, parenterally, subcutaneously, intramuscular, intravenously or intracerebrally. The preferred route of administration would be parenterally in a daily dosage of the compound for an adult of about 0.01-5000 mg, preferably 1-1500 mg per day. Preferably, the medicament is administered in a dosage of between 30 mg/day and 2000 mg/day, preferred between 100 mg/day and 1600 mg/day, most preferred between 300 to 800 mg/day. The suitable dosage can be presented as a single dosage or as divided dosages, in suitable intervals, for example as two, three, four or more sub-dosages per day. Suitable dosages can be readily obtained by a person of skill using routine experiments, and can be based on factors, such as, for example, the concentration of the active ingredient, the body weight and age of the patient and other patient- or active ingredient-related factors.

Pharmaceutical compositions in general are administered in an amount, which is effective for the treatment or prophylaxis of a specific state or states. The initial dosing in a human is accompanied by clinical monitoring of symptoms, the symptoms of the selected state. In general, the compositions are administered in an amount of active agent of at least about 100 μg/kg body weight. In most cases they are administered in one or more dosages in an amount not exceeding about 20 mg/kg body weight per day. Preferred in most cases is a dosage of about 100 μg/kg to about 5 mg/kg body weight per day.

The fields of use for medicaments on the basis of the present invention are manifold and relate to disorders and/or diseases that are mediated by Fc-gamma-receptor activating antibodies in the field of oncology, infectious diseases, the musculo-skeletal system, endocrine and/or metabolic functional disorders, hematology, respiratory diseases, diseases of the CNS and immunological diseases and inflammatory diseases.

A still further aspect of the present invention then relates to a method for detecting Fc-gamma-receptor activating antibodies in a sample, comprising a method for measuring the strength of the interaction between the constant parts of a monoclonal antibody and an Fc-receptor according to the invention, wherein an expression of IL-2 is an indication for the presence of Fc-gamma-receptor activating antibodies in the sample.

A sample in the sense of the present invention can be any potentially Fc-gamma-receptor activating antibodies containing sample, such as, for example, whole blood, serum (preferred) or fractions or components thereof Furthermore, the sample can be a sample containing recombinantly produced antibodies or parts comprising Fc-regions, e.g. a sample in a buffer or an antibody fraction which has been obtained in another manner from a hybridoma culture.

Preferred is a method for detecting Fc-gamma-receptor activating antibodies in a sample according to the invention, wherein the activating antibody is an autoimmune antibody.

Further preferred is a method according to the present invention, wherein the sample is analyzed in the context of an oncological disease, an infectious disease, an autoimmune disease, a disease of the musculo-skeletal system, an endocrine and/or metabolic functional disorder, a hematological disease, a respiratory disease, diseases of the CNS and/or an immunological disease

The inventors have developed a novel test system, in order to detect and to quantify virus-immune-IgG which can activate FcγRs after the opsonization of infected target cells. Using a comprehensive set of FcγR-ζ chimeric receptors, the binding of poly- or monoclonal IgGs to virus-infected target cells was translated into a IL-2 secretion of BW 5147 cells. The separation of IgG-activated single FcγRs within the global virus-specific IgG response showed surprising differences in the composition of IgG responses between individuals.

Despite the fact that the IgG binding to the FcγRs during the induction and the effector-phase of immune responses is decisive, only very limited simple methodology exists, in order to measure these immune responses in vitro. This limitation can be attributed to the lack of simple, reliable and standardized tests, which possibly contributes to the relative negligence of a development regarding FcγR activating antibodies. ADCC represents a surrogate for FcγRIII-mediated IgG responses, nevertheless, the use of primarily heterogeneous effector cell population, such as, for example, PBMC or isolated and in vitro propagated NK cell populations, in ADCC Tests often generates problems due to the variability in the FcγR and NK-cellular marker expression, and the fluctuating activation status of effector cells. These imponderabilities render the interpretation of test results difficult. As a consequence, using the ADCC tests the determination of immune-IgG titers is neither reliable nor sensitive and for these reasons unsuitable for the routine diagnostic.

The present test system according to the invention has several advantages compared to traditional ADCC tests: i) a homogenous effector cell population, which only expresses one strictly defined FcγR exprimiert, which eliminates the need for cell preparations from cell donors: ii) a high intra- and inter-test reproducibility, based on a constant and unlimited effector cell population and available immune IgG standards; iii) a comprehensive panel of FcγR for measuring specific responses; iv) low detection limits; and v) an excellent sensitivity of the test, generating quantifiable data. From a practical point of view: the BW FcγR-ζ effector cells can be kept rather easily, and the test method does not require radioactive isotopes.

The detection of virus-immune-antibodies in classical PRNT is based on only very few anti-genic determinants, that are present on the virion-surface, and are associated with the critical steps of the viral attachment and the entry into the target cell. In contrast thereto, the main fraction of virus-immune-IgG that is produced in response to infection is directed against non-neutralizing epitopes that are formed by structural surface proteins, non-structural viral polypeptides, and internal proteins of the virion, whereby all of them lack the neutralizing activity. These IgG specificities comprise the main fraction of biophysically binding antibodies that react in ELISA tests. The targets of the IgGs as detected in the BW FcγR-ζ tests represent a discrete class of viral antigens, which are characterized by their infected host cell-surface disposition and thus include both structural as well as non-structural transmembrane proteins, depending from the protein content of a particular virus. Nevertheless, the novel test principle according to the invention detects only those IgGs, which can activate a defined FcγR, when bound to a viral determinant in its native conformation, which is presented on the surface of an infected cell. Several characteristics will enhance or limit this ability, such as the subclass of the bound IgG molecule, the number and the density of the epitopes on the surface of the target cell, the fluidity of the membrane of the target cell, which controls the IgG dislocation after FcγR ligation, and the conformational re-arrangement of the immune complex as formed. The result that Palivizumab and HCMV-immune IgG CD16, CD32 and CD64 can only activate with very high efficiency when they first have bound to their epitopes, but not in a monomeric conformation, illustrates the enormous conformational influence of the antigen auf den recognition process that leads to an activation of the FcγR.

In contrast to neutralizing virion-epitopes, very little is known about the antigenic determinants, that are recognized by the IgG clonotypes that are covered by the BW FcγR-ζ test. Thus, a direct use of the test system is the identification and mapping of dominant viral FcγR-activating epitopes, that are presented on the surface of infected cells by means of monoclonal as well as natural polyclonal IgGs. Certain viruses, such as, for example influenza or HIV, replicate under a rigorous selection pressure of neutralizing IgGs, leading to a viral immune-escape. The tests as developed according to the invention are used in order to show, whether these viruses use the same or essentially the same evasive mechanisms in case of epitopes that are recognized by IgG specificities, which activate FcγRs. When then adjusted to Fc receptors that are specific for other Ig subclasses, such as, for example IgA or IgM, the test according to the invention becomes suitable for the detection of immune IgA or IgM responses. In addition to the versatile use as a novel immune test-tool for medical virology, parasitology or bacteriology, the test principle according to the invention can be used in many areas of immune diagnostic and development.

During the last years, numerous evidence has been collected that the involvement of defined FcγRs is of extreme importance for the therapeutic effects of tumor specific IgG, anti-inflammatory effects of intravenous Ig and IgG-mediated autoimmune diseases. This knowledge had stimulated approaches to develop IgG as a therapeutic tool for the immune-intervention. The new test system of the present invention is useful in the improvement of the effectivity of such IgGs, since optimal IgG-Fc-FcγR interactions can be identified.

Specific embodiments of the present invention are illustrated based on the Figures and the examples, nevertheless, without being limited to these. All of the references as cited are herewith incorporated by reference in their entireties.

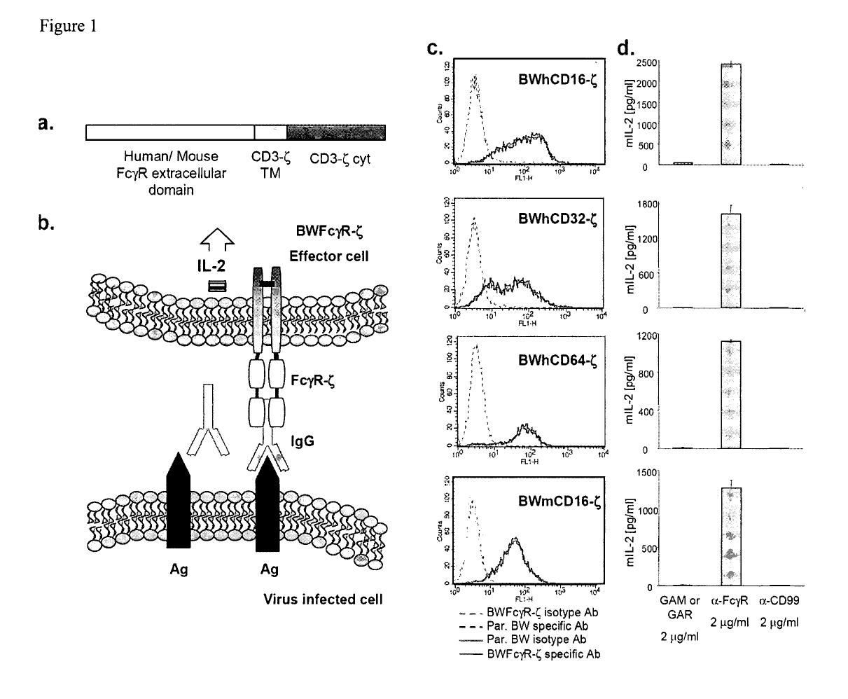

FIG. 1

Setup of FcγR activation assay. (A) Schematic representation of FcγR-ζ chimeras. Extracellular domains of human or mouse-FcγR (weiβ) were fused to the transmembrane domain (light grey) and the intra-cytoplasmatic tail of mouse CD3ζ (dark grey). (B) Schematic representation of the test principle. FcγR-ζ ligation by immune-IgG causes the mIL-2 secretion in BW FcγR-ζ effector cells. (C) Detection of FcγR chimeras auf BW 5147 transfectants using FACS. Dark grey continuous line: anti-FcγR-FITC mAb. Dark grey dashed line: Transfectant without Ab. Light grey dashed line: Parenteral cell with anti FcγR-FITC mAb. Light grey continuous line: secondary Ab GAM-FITC. (D) Cross-linking experiments with mAb, directed against the ectodomain of the FcγRs, in order to show the intact signal transduction. GAM or goat anti rat IgG (GAR) were coated in 96-well cell culture plates at a concentration of 2 ug/ml. After blocking and washing mouse mAbs specific for human CD16-A/B, human CD32, human CD64 and rat anti-mouse CD16/CD32 were added. As a negative control mAb anti-human CD99 was used. After removal of unbound antibodies 200.000 BW FcγR-ζ transfectants per well were added. The mIL-2 secretion was determined after 16 h of incubation.

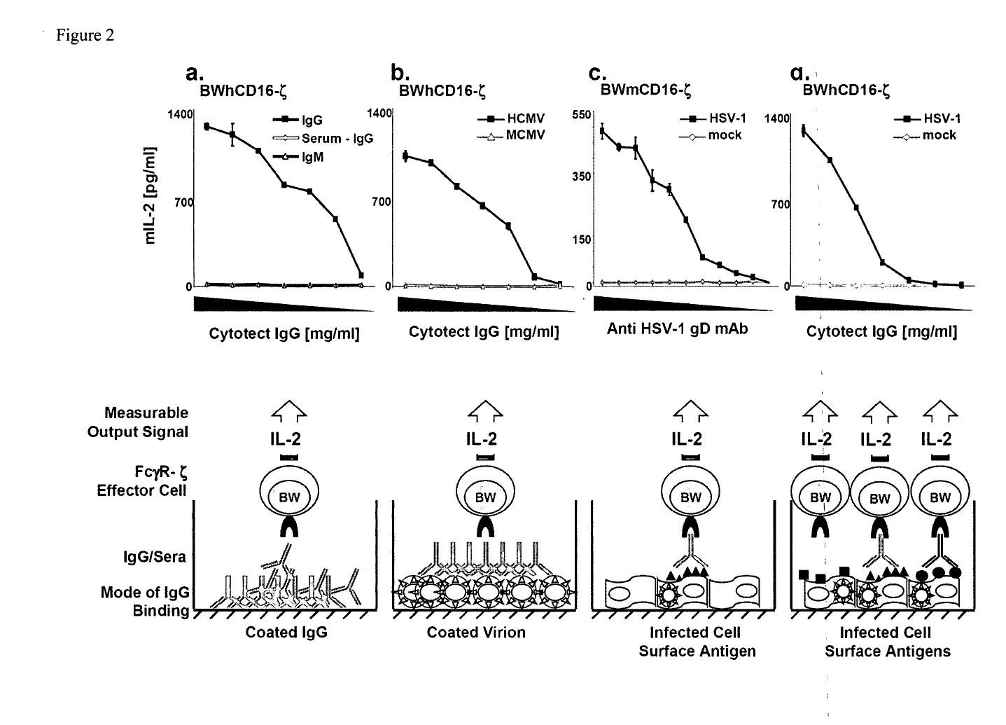

FIG. 2

IgG mediated activation of BW FcγR-transfectants. (A) Cytotect® or virion preparations were coated in binding buffer (0.1 M Na₂HPO₄ pH 9.0). After blocking and washing serial dilutions of Cytotect® IgG (10 mg/ml to 0.1 mg/ml) were added to coated virions, and incubated over 30 minutes at 37° C. After removal of unbound antibodies 100.000 cells of BW FcγR-ζ transfectants were added per well. MRC-5 cells were infected with 2 pfu per cell of HSV-I. 24 h post infection serial dilutions of (C) anti-gD HD1 mAb or (D) Cytotect® were added, and incubated for 30 minutes at 37° C. After removal of unbound antibody 100.000 BWFcγR-ζ transfectants were added per well. mIL-2 was detected after 16 h using ELISA. A schematic diagram of the IgG-dependent activation of BW FcγR-ζ transfectants is shown below the charts.

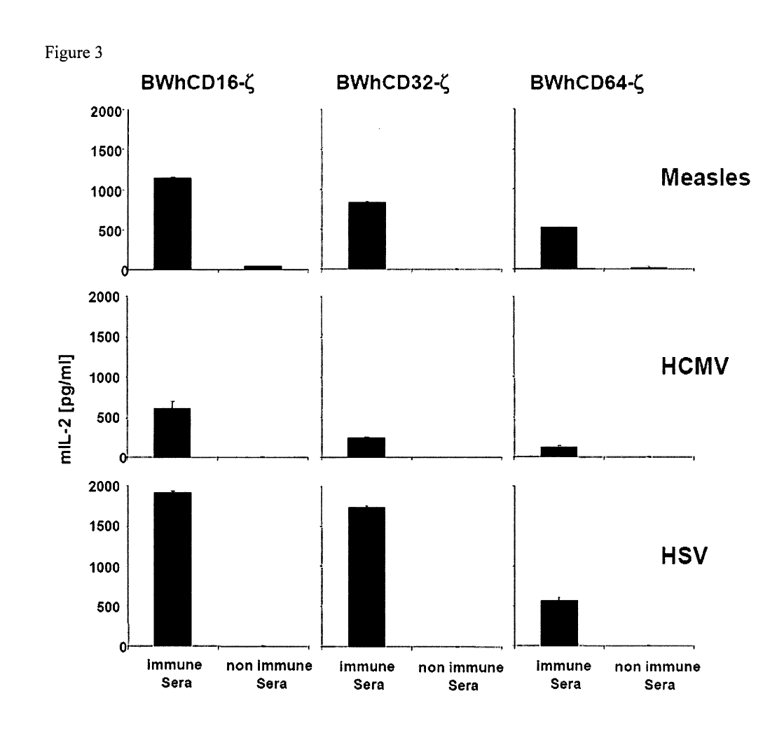

FIG. 3

Antigen specificity of FcγR-ζ activation by immune-IgG. MRC-5 cells were infected with HCMV (2 pfu/cell) for 72 h, Vero cells were infected with HSV (2 pfu/cell) for 24 h or MV (2 pfu/cell) for 72 h, before opsonization with IgG of pooled human sera took place. ELISA-reactive sera were compared to ELISA of non-reactive sera at 2 mg/ml of IgG concentration. After washing, BW FcγR-ζ effector cells were added in an E:T ratio of 20:1, and the cultures were incubated for 16 h. mIL-2 was measured using ELISA.

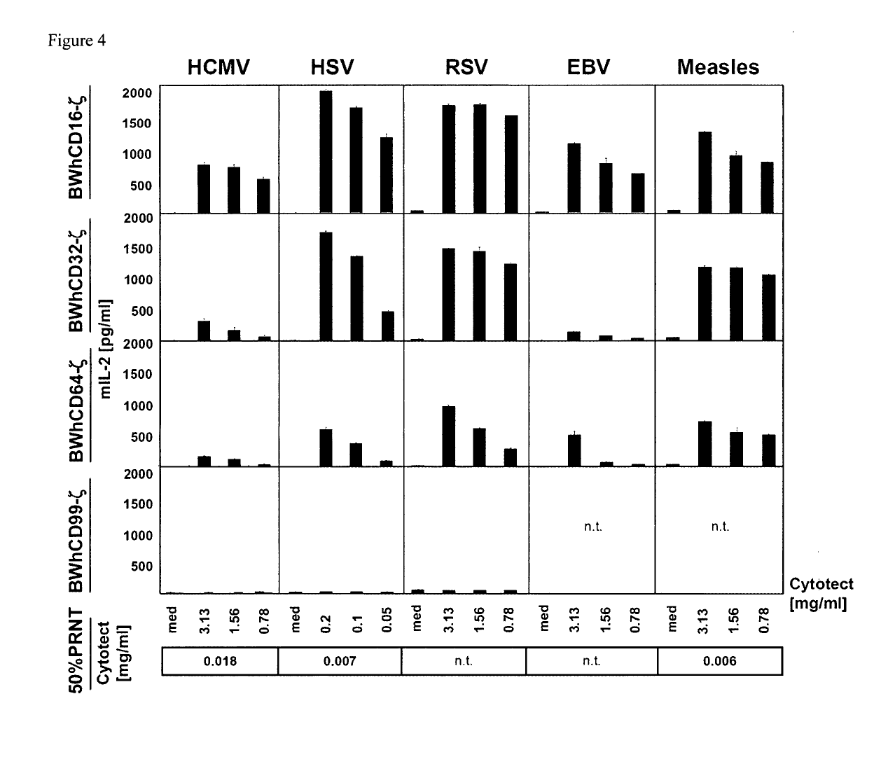

FIG. 4

Detection of virus-immune IgG activating host-FcγRs in a large human IgG preparation. MRC-5 cells were infected with HCMV (2 pfu/cell) for 72 h, Vero cells were infected with HSV (2 pfu/cell) for 24 h and MV (2 pfu/cell) for 72 h, Hep-2 cells were infected with RSV (2 pfu/cell) for 72 h, the EBV-infected B95.8-cell line was subsequently opsonized with the given Cytotect® concentrations. After removal of unbound IgG, BW FcγR-ζ effector cells were added in an E:T ratio of 20:1, and the cultures were incubated for 16 h. The amount of m1L-2 was determined using ELISA. Identical dilutions of Cytotect® were used for all viruses, with the exception of HSV-1. n.t. not tested. The PRNT titer of Cytotect® (bottom) are shown as determined for the viruses as indicated.

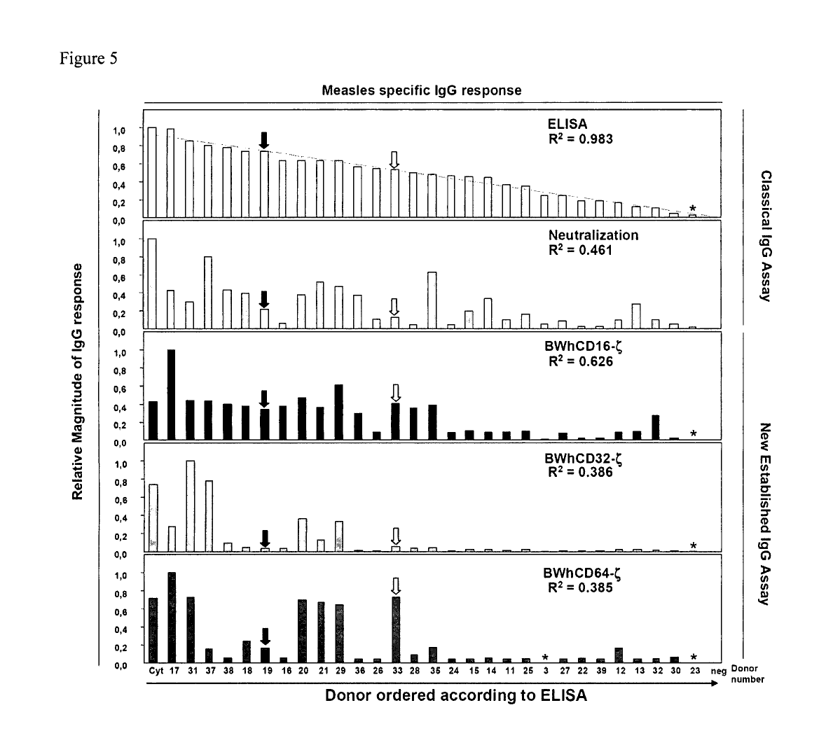

FIG. 5

Immunograms: Analysis of heterogeneous MV IgG reaction patterns of individual sera. Individual donor sera were analyzed with the tests as indicated for MV-specific IgG responses. The order of the samples arranged according to the relative magnitude of the response as measured using ELISA. Donor No. 19 and No. 33 are each highlighted by black and white arrows. Donors highlighted by an asterisk were found below the detection limit of each test. Linear correlation values (R²), determining the linearity for each test, are given. Cyt Cytotect®, neg. a negative donor.

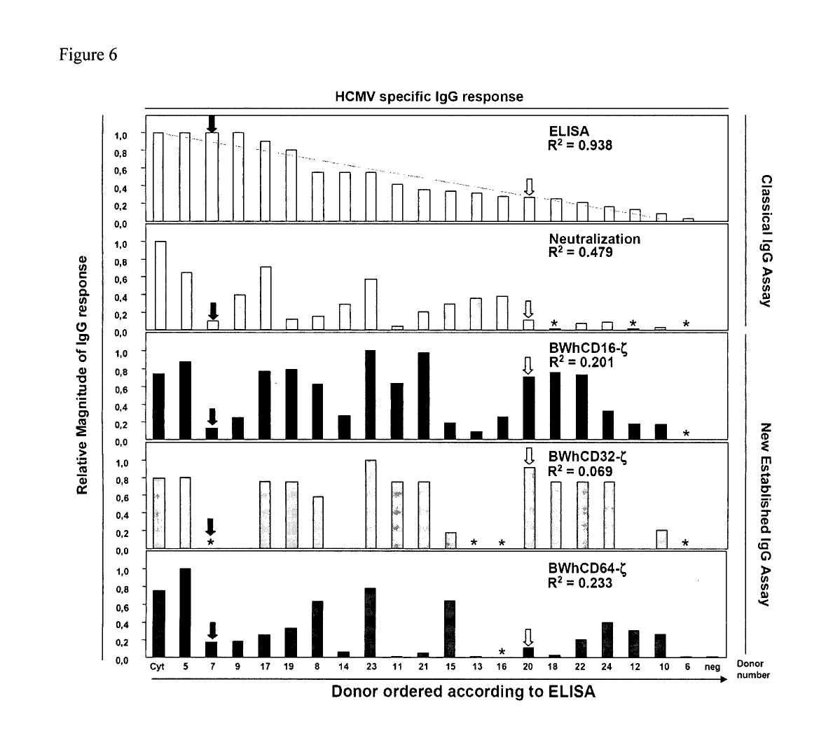

FIG. 6

Immunograms: Analysis of HCMV IgG reaction patterns of individual sera. Individual donor sera were analyzed with the tests as indicated for HCMV-specific IgG responses The order of the samples arranged according to the relative magnitude of the response as measured using ELISA. The profiles as seen in the hCD16-ζ and hCD32-ζ tests were astonishingly consistent. Donor Nr. 7 and Nr. 20 are each highlighted by black and white arrows. Donors highlighted by an asterisk were found below the detection limit of each test. Linear correlation values (R²), determining the linearity for each test, are given. Cyt Cytotect®, neg. a negative donor.

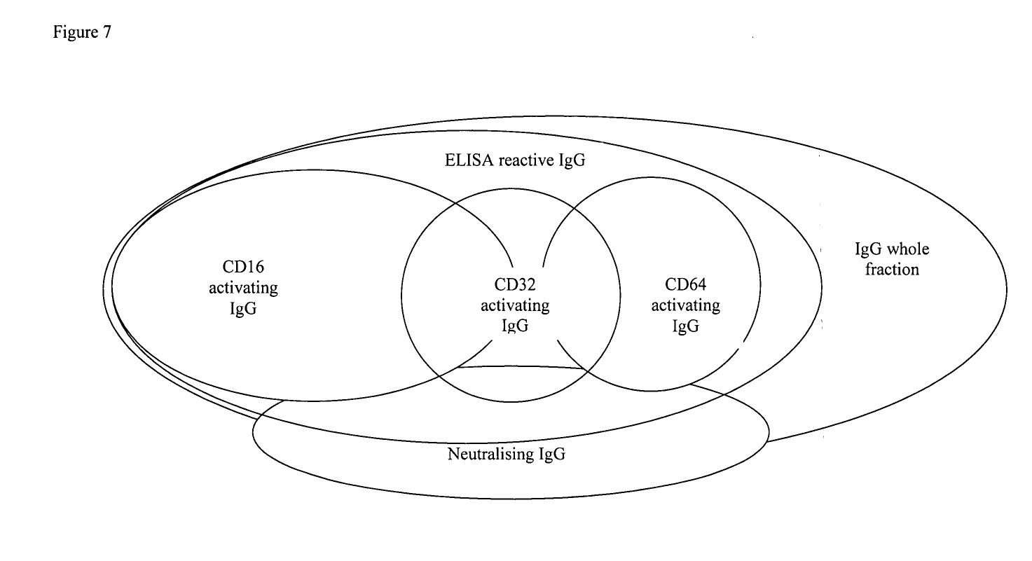

FIG. 7

Sub-composition of the virus-specific IgG response (“immunogram”). As a fraction of the overall amount of serum IgGs, the pool of virus-immune Ab can be detected using ELISA in dependence from the array of viral antigens that are represented in the test, and the biophysical binding characteristics of immune-IgGs. Within the ELISA-reactive IgG-fraction, some virus-immune-IgG clonotypes have distinct functional properties, i.e. virion-neutralization or activation of FcγRs (CD16, CD32 or CD64) after recognition of viral epitopes on the cellular surface. Some IgGs can exhibit overlapping functional properties.

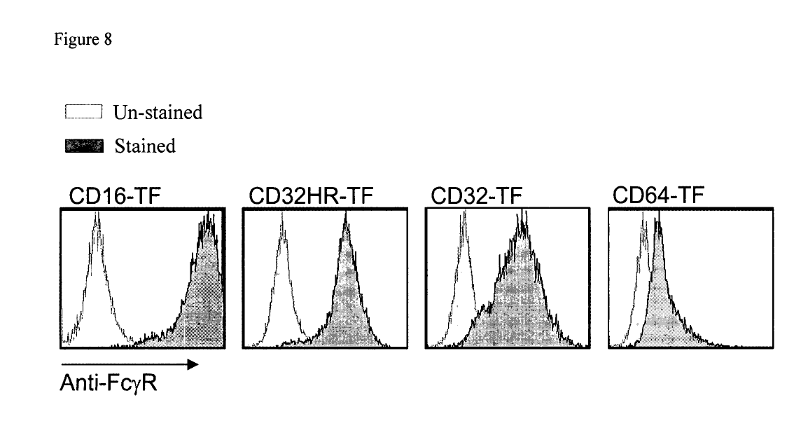

FIG. 8

Flowcytometric analysis of FcγR-expressing transfectants. 1×10⁶ FcγR-expressing cells of the stable transfectants CD16-TF, CD32HR-TF, CD32-TF, and CD64-TF were stained with fluorescently labeled antibodies directed against CD16, CD32 or CD64, and analyzed using a flow-cytometry. The data are representative for three independent stainings.

### EXAMPLES

In the following examples, the method according to the invention is tested in connection with an immune response of antibodies against viral antigens. It should be understood that the method of the invention can also be used for antibodies that recognize other antigens.

Both structural as well as non-structural viral proteins induce antigen-specific IgG responses. The detection of virus-specific IgGs is essential for diagnostic purposes in many clinical applications. The presence of immune-IgGs is detected regularly by prototypic in vitro tests, such as, for example, ELISA (enzyme-linked immunosorbent assay), cell-based immunofluorescence assays, immunoblots, hemagglutination-inhibition and virus neutralization tests. Nevertheless, only the latter method provides a direct information about a biological effector function of the immune-IgG. Only a fraction of the virus-specific IgGs affects a direct antiviral activity through inhibiting of the infectivity of virions, complement activation or FcγR activation. Neutralizing antibodies inhibit the viral binding to entry receptors of the target cell or prevent the viral fusion with the host membrane. Nevertheless, many of the epitopes that are exposed on the surface of the virion or cell are non-neutralizing, when they bind IgGs. Sub-fractions of IgG can cause additional immune functions by complement activation, an increase the phagocytosis (opsonization), and cause ADCC (antibody-dependent cellular cytotoxicity). Observations in B cell deficient mice showed a prominent role for IgG in the control of the viral replication and re-infection. The neutralization of the infectivity of the virus by Abs is an effective way, in order to stop the infection, which explains the vaccine-mediated protection of the neutralizing IgGs. The failure of adoptively transferred neutralizing Abs to protected against specific viral diseases, which are known to be sensitive against the Ab immune control, leads to the assumption, that non-neutralizing Abs can significantly contribute to a protection.

It is assumed that the FcγR-mediated immune responses, including ADCC and the cytokine-release, are essential components of the response against pathogens and in particular viruses. ADCC leads to a lysis of virus-infected cells, when immune-IgG opsonized target cells are recognized by FcγR carrying cytotoxic effector cells, and activate them. Different FcγRs contribute to classical ADCC responses, it was shown for CD16+NK cells that they induce the process most efficiently. Different methods to measure ADCC in vitro were developed (Clémenceau B, Congy-Jolivet N, Gallot G, Vivien R, Gaschet J, Thibault G, Vié H. Antibody-dependent cellular cytotoxicity (ADCC) is mediated by genetically modified antigen-specific human T lymphocytes. Blood. 2006 Jun. 15; 107 (12):4669-77. Epub 2006 Mar. 2; Gómez-Román V R, Florese R H, Patterson L J, Peng B, Venzon D, Aldrich K, Robert-Guroff M. A simplified method for the rapid fluorometric assessment of antibody-dependent cell-mediated cytotoxicity. J Immunol Methods. 2006 Jan. 20; 308 (1-2):53-67. Epub 2005 Nov. 28; Kantakamalakul W, Pattanapanyasat K, Jongrakthaitae S, Assawadarachai V, Ampol S, Sutthent R. A novel EGFP-CEM-NKr flow cytometric method for measuring antibody dependent cell mediated-cytotoxicity (ADCC) activity in HIV-1 infected individuals. J Immunol Methods. 2006 Aug. 31; 315 (1-2):1-10. Epub 2006 Jul. 17; Lichtenfels R, Biddison W E, Schulz H, Vogt A B, Martin R. CARE-LASS (calcein-release-assay), an improved fluorescence-based test system to measure cytotoxic T lymphocyte activity. J Immunol Methods. 1994 Jun. 24; 172 (2):227-39), nevertheless, many tests suffer from the disadvantage that they use inhomogeneous effector cells, which strongly vary in type and density of the FcγR expression. In addition, these effector cells are difficult to prepare and to keep in culture, and thus are available only in limited amounts. These problems could be overcome, if a clonal effector cell-population would be used that expresses a defined FcγR. Thus, a selection of novel effector cells was established in order measure the capacity of (in this case exemplary and preferred) virus-specific IgG to activate defined types of FcγRs. The tests include the co-culturing of virus-infected target cells with BW 5147 transfectants, which stably express a chimeric FcγR, in the presence of poly- or monoclonal immune-IgGs. The FcγR chimera carry the extracellular domain of defined FcγRs, i.e. human CD16, CD32, CD64 and mouse CD16, which are fused to the transmembrane and intracellular tail domain of the mouse CD3-ζ chain. After activation of the chimeric FcγRs, mouse IL-2 is secreted and can readily measured. Thus, the test system quantifies the capacity of virus-immune-IgGs to activate FcγRs in a receptor type-specific manner, after the recognition of a naturally formed epitope, which is presented on the surface of infected target cells.

Using a comparable and comprehensive set of type-specific FcγR tests with a large pool of polyclonal human immune-IgGs, substantial differences in the activation of FcγRs between different viruses could be shown. In addition, individual types of FcγRs were activated to different extends; the CD16 responses identified as being the strongest. Thereafter multiple human sera were tested for their individual capacity, subsequent to opsonisation to Virus-infected cells, to activate FcγRs. Here, only a low correlation between the global IgG response having specificity for measles virus (MV) and human cytomegalovirus (HCMV) was found, when they were measured using ELISA, similar to the comparison with neutralizing IgG against the respective virus.

### Cloning of the FcγR-ζ Constructs

The cloning of cDNAs for human FcγRs coding for CD16a (higher affinity variant with a valine at position 158 (Bowles J A, Weiner G J. CD16 polymorphisms and NK activation induced by monoclonal antibody-coated target cells. J Immunol Methods. 2005 September; 304 (1-2):88-99)), CD32a and CD64, was described previously (Allen J M, Seed B. Isolation and expression of functional high-affinity Fc receptor complementary DNAs. Science. 1989 Jan. 20; 243 (4889):378-81; Stengelin S, Stamenkovic I, Seed B. Isolation of cDNAs for two distinct human Fc receptors by ligand affinity cloning. EMBO J. 1988 April; 7 (4):1053-9).

In order to clone mouse CD16, cDNA was prepared from C57BL/6 splenocytes. The extracellular part of all FcγRs was cloned by means of PCR using suitable primer pairs containing sites for restriction endonucleases.

The FcγR-ζ Genes were subcloned into the pcDNA3.1 expression vector (Invitrogen Corp, Carlsbad, Calif., USA) and used to generate stable BW transfectants. Human CD16-ζ and hBW CD99-ζ transfectants were described previously (Mandelboim O, Malik P, Davis D M, Jo C H, Boyson J E, Strominger J L. Human CD16 as a lysis receptor mediating direct natural killer cell cytotoxicity. Proc Natl Acad Sci USA. 1999 May 11; 96 (10):5640-4; Mandelboim O, Lieberman N, Lev M, Paul L, Arnon T I, Bushkin Y, Davis D M, Strominger J L, Yewdell J W, Porgador A. Recognition of haemagglutinins on virus-infected cells by NKp46 activates lysis by human NK cells. Nature. 2001 Feb. 22; 409 (6823):1055-60).

### IgG Dependent Activation of BW FcγR-ζ Transfectants

In order to assess the activation of BW FcγR-ζ cells by monomeric IgG and by immune complexes, human IgG (Cytotect®) and purified HCMV and MCMV virion preparations were coated onto plates. To measure the reactivity of BW FcγR-ζ transfectants in response to IgG, if a native viral cell-surface antigen is recognized, a co-culture-test was performed. AB-induced signalling through the chimeric mouse CD16 FcγR-ζ receptor was tested using a stepwise concentration gradient of a mouse mAb HD-I, which recognizes a surface epitope of HSV-1 gD after incubation with HSV-1 infected MRC-5 fibroblasts. In other experiments mock- and virus-infected cells were incubated with a two times serial dilution of human sera in complete D-MEM for 30 min at 37° C. in a 5% CO₂ atmosphere. The cells were washed three times in complete medium in order to remove the unbound IgG, before co-culturing with BW FcγR-ζ transfectants for 16 h. In standard experiments all tests were performed in triplicates, and the relation of the effectors (BW FcγR-ζ Transfektanten) to the virus-infected target cell was 20:1. Subsequent to co-culturing for 16 h at 37° C. in a 5% CO₂ atmosphere, supernatants were diluted 1:2 in ELISA sample buffer (PBS with 10% FBS and 0.1% Tween 20), and mIL-2 was measured using ELISA. In the case the BW FcγR-ζ test was performed for suspension-cells, V-bottom cell culture dishes were used. 1×10⁴ EBV-infected B95.8 cells were added to prior diluted sera. After incubation of the cells for 30 min at 37° C. in an atmosphere of 5% CO₂, three washing steps were applied to remove unbound IgG. Co-culturing of BW FcγRζ transfectants and the detection of IL-2 was conducted as described above.

In order to assess the specificity, sensitivity, negative and positive predictive values and the test efficiency of BW hCD16-ζ, BW hCD32-ζ and of the BW hCD64-ζ tests, groups of sera of 38 donors having an unknown MV-sera status were tested in an IgG- and an IgM-MV specific ELISA (Enzygnost, Dade Behring, Germany). Only MV-IgM negative sera were analysed in a MV plaque reduction-neutralisation test (PRNT) and the BW FcγR-ζ activation test. Furthermore, a series of 46 healthy donors with unknown HCMV sera status was analysed in a HCMV IgG and IgM ELISA, and in tested PRNT using HCMV AD169. HCMV-IgM negative sera were analysed in the BW hCD16-ζ, BW hCD32-ζ and the BW hCD64-ζ test. Positive values in the BW FcγR-ζ test were defined as the serum dilution that is necessary to reach the exclusion-value. The exclusion-value was determined from the activation of the BW transfectants after co-culturing with infected cells and sera from 15 different sero-negative donors. Three SD (standard deviations) were added to the arithmetic mean of the through 15 sero-negative donors induced mIL-2, and this value defined as exclusion-value.

Generation of stable FcγR-ζ BW5147 transfectants and detection of mIL-2

TCRαβζ negative BW 5147 thymoma cells were transfected with pcDNA3.1 constructs using Superfect (Qiagen GmbH, Hilden, Germany), which code for FcγR-ζ chimeras. The activation of the cytoplasmatic domain of the TCR-ζ-chain is sufficient to elicit the IL-2 secretion (Irving B A, Weiss A. The cytoplasmatic domain of the T cell receptor zeta chain is sufficient to couple to receptor-associated signal transduction pathways. Cell. 1991 Mar. 8; 64 (5):891-901). Stable BW transfectants were selected at a concentration of 3 mg/ml Geneticin (G418) (Sigma-Aldrich, Germany). After 4 weeks, the FcγR surface expression was tested using FACS (Excalibur, Becton Dickinson, California, USA) using mouse anti-human CD16-FITC (Miltenyi Biotec GmbH, Bergisch Gladbach, Germany), mouse anti-human CD32-FITC (BD Pharmigen™, Erembodegem, Belgium), mouse anti-mouse CD64 (BD Pharmigen™, Erembodegem, Belgium), rat anti-mouse CD16/CD32 (BioLegend, Inc, San Diego, Calif.), goat anti-rat IgG-FITC (Sigma-Aldrich, Germany) and mouse Fc-FITC (Rockland Immunochemicals, PA. USA). The result of the FACS analysis is depicted in FIG. 8. For all cell lines a stable expression of the respective FcγR-ζ on the cell surface was detectable.

Secreted mIL-2 from BW FcγR-ζ transfectants was measured using ELISA using the capture AB JES6-1A12 and the biotinylated detection-antibody JES6-5H4 (BD Pharmigen™, Erembodegem, Belgien). The detection limit was at about 3 pg/mL IL-2.

In order to detect the maximal levels of mIL-2 induction of the transfectants, FcγR-ζ-cross-linking experiments were performed. Goat anti-mouse IgG (Dianova, Germany) or goat anti-rat IgG (Dianova Deutschland) were coated in 96-well cell culture plates at a concentration of 1 μg/mL in binding buffer (0.1 M Na₂HPO₄ pH 9.0). After blocking and washing, mouse mAb, specific for human CD16-A/B (Santa Cruz Biotechnology, USA), human CD32 (Santa Cruz Biotechnology, USA), human CD64 (Ancell Corporation, USA), and rat anti-mouse CD16/CD32 (BioLegend, Inc, San Diego, Calif.) were added. Anti-human CD99 mAb was used as a negative control. After removal of unbound antibodies 200.000 BW FcγR-ζ transfectants were added per well. The mIL-2 secretion was determined after 16 h incubation.

### The Stable Expression of FcγR-ζ Chimeras Mediate the Activation of BW FcγR-ζ Transfectants

Mouse BW 5147 thymoma cells lacking the TCR α, β and ζ chains were transfected with constructs encoding for chimeric FcγR proteins, wherein the extracellular part of human CD16, human CD32, human CD64 and mouse CD16, respectively, was fused to the transmembrane and intracellular tail domain of the CD3ζ chain (FIG. 1A). Thus, the signaling cascade as initiated by the ligand binding to the chimeric FcγR mimics the TCR activation (FIG. 1B). The expression of chimeric FcγRs by G41U resistant cells was detected by cell surface staining and FACS analysis. All chimeric FcγRs were detected in high densities on the plasma membrane of BW cells (FIG. 1C). in order to detect an intact signal transduction by the hCD16-ζ, hCD32-ζ, hCD64-ζ and mCD16-ζ receptors, mAbs directed against each of the FcγR ectodomains were used in cross-linking experiments. All transfectants responded efficiently and secreted high levels of mIL-2, whereas an mAb directed against CD99 IL-2 did not induce (FIG. 1D). A hCD99-ζ chimera served as an additional control. The dosage-dependent release of mIL-2 from BW hCD99-ζ transfectants was achieved by incubation with anti-human CD99 mAb, but not when the BW hCD99-ζ transfectants were treated with an mAb directed against human CD16. All BW transfectants thus responded efficiently, when cross-linked by specific mAbs that were directed against the extracellular domain of the chimeric FcγRs.

### Universal Applicability of the Tests, in Order to Detect Virus-Specific IgGs

The data as given here showed that FcγR-ζ transfectants react to immune-IgGs which are bound to epitopes, which are presented on HSV- and RSV-infected cells as well as HCMV virions. In order to detect, whether the FcγR-ζ activation tests can also be used in order to detect immune-IgG against other human pathogenic viruses and other antigens, the analysis was expanded to target cells, which were infected with the β-herpes virus HCMV, the γ-herpes virus EBV, and the paramyxoviridae family member MV, when these were opsonized with different concentrations of Cytotect®. A true antigen specificity of virus detection was first ensured using pooled human sera, which were non-reactive in ELISAs, wherein immune-IgG against the particular viruses (non-immune sera) that did not cause FcγR-ζ responses were detected, in contrast to pools of ELISA-reactive (immune) sera (FIG. 5).

As shown in FIG. 6, immune-IgG, which is present in Cytotect®, was able to activate BW hCD16-ζ cells, BW hCD32-ζ cells, and BW hCD64-ζ, but no BW hCD99-ζ cells after co-culturing with target cells, that were infected with viruses of the test panel, nevertheless, with different efficiency. In general, the activation of BW hCD16-ζ cells by Cytotect® was more efficient than the activation of BW hCD32-ζ, whereas the stimulation of BW hCDCD64-ζ transfectants was the weakest. Essential differences between viruses were observed, since hCD32-ζ was activated markedly weaker after co-culturing with HCMV and EBV-infected cells, compared with HSV or both paramyxoviruses. The titers for HSV-immune-IgG, which were measured for the activation of all FcγR-ζ receptors, compared to all other viruses were the highest in the Cytotect® preparation. In contrast to the FcγR activating IgGs the HSV-neutralizing capacity of Cytotect® was found in a similar range, compared with MV and HCMV, where the titers as measured using 50% PRNT were markedly higher (RSV: 0.025 mg/ml; MV: 0.018 mg/ml; HCMV: 0.066 mg/ml; FIG. 6). In summary, it was shown using the FcγR-ζ-based tests that they can be used for all of the viruses as tested, i.e. very broadly, and thus in principle can be used for other surface antigens. The titer of virus-specific IgG that can induce FcγR responses in a large pool of human sera, depends from the respective virus and the type of FcγR. No correlation with the concentration of neutralizing IgG can be found (see following tables).

TABLE 1
Correlation between the different tests as used fort he detection of MV-IgG

| | ELISA | PRNT | BWhCD16-ζ | BWhCD32-ζ | BWhCD64-ζ |
|---|---|---|---|---|---|
| ELISA | 1.00 | | | | |
| PRNT | 0.76 | 1.00 | | | |
| BWhCD16-ζ | 0.94 | 0.68 | 1.00 | | |
| BWhCD32-ζ | 0.93 | 0.67 | 0.98 | 1.00 | |
| BWhCD64-ζ | 0.86 | 0.58 | 0.91 | 0.92 | 1.00 |

TABLE 2
Efficiency of the FcγR-ζ based test compared with MV IgG ELISA

| | PRNT | BWhCD16-ζ | BWhCD32-ζ | BWhCD64-ζ |
|---|---|---|---|---|
| Sensitivity | 100.0 | 100.0 | 100.0 | 96.6 |
| Specificity | 90.0 | 100.0 | 100.0 | 90.9 |
| Negative | 100.0 | 100.0 | 100.0 | 90.9 |
| Predictive Value | | | | |
| Positive | 96.7 | 100.0 | 100.0 | 96.6 |
| Predictive Value | | | | |
| Test Efficiency | 97.4 | 100.0 | 100.0 | 95.0 |

TABLE 3
Correlation between the different tests as used for the detection of HCMV-IgG

| | ELISA | PRNT | BWhCD16-ζ | BWhCD32-ζ | BWhCD64-ζ |
|---|---|---|---|---|---|
| ELISA | 1.00 | | | | |
| PRNT | 0.97 | 1.00 | | | |
| BWhCD16-ζ | 0.85 | 0.87 | 1.00 | | |
| BWhCD32-ζ | 0.85 | 0.88 | 0.84 | 1.00 | |
| BWhCD64-ζ | 0.79 | 0.77 | 0.68 | 0.75 | 1.00 |

TABLE 4
Efficiency of the FcγR-ζ based tests compared with
HCMV IgG ELISA

| | PRNT | BWhCD16-ζ | BWhCD32-ζ | BWhCD64-ζ |
|---|---|---|---|---|
| Sensitivity | 90.5 | 95.2 | 81.0 | 95.2 |
| Specificity | 100.0 | 92.3 | 92.3 | 76.9 |
| Negative | 92.9 | 96.0 | 85.7 | 95.2 |
| Predictive Value | | | | |
| Positive | 100.0 | 90.9 | 89.5 | 76.9 |
| Predictive Value | | | | |
| Test Efficiency | 95.7 | 93.6 | 87.2 | 85.1 |

It should be understood that the features of the invention as herein described and disclosed can not only be embodied in the respective combination as explicitly described, but can also be realized in an individualized manner without departing from the intended scope of the present invention.

1. A recombinant expression vector, comprising sequences for the recombinant expression of at least one fusion protein on the surface of a mammalian cell, wherein the fusion protein comprises a) an extracellular part of a mammalian Fc-receptor, b) a transmembrane region of a zeta-chain, and c) an intracellular signaling domain of a T-cell receptor zeta-chain.

2. The recombinant expression vector according to claim 1, wherein said mammalian cell is a human- or mouse-zeta-chain-deficient lymphoma cell.

3. The recombinant expression vector according to claim 1, wherein said receptor is selected from CD16, CD32, and CD64.

4. The recombinant expression vector according to claim 1, wherein said Fc-receptor-zeta-chain is fused with an Fc-receptor for another Ig-subclass.

5. A method for producing a recombinant mammalian lymphoma cell, comprising transfecting a zeta-chain-deficient mammalian lymphoma cell with an expression vector according to claim 1, and expressing at least one fusion protein on a surface of the cell.

6. A recombinant mammalian lymphoma cell, produced according to claim 5.

7. The recombinant mammalian lymphoma cell according to claim 6, wherein said cell is selected from a human- or mouse-lymphoma cell.

8. The recombinant mammalian lymphoma cell according to claim 6, wherein said receptor is selected from CD16, CD32, and CD64.

9. A method for measuring the strength of an interaction between constant parts of a monoclonal antibody and an Fc-receptor, comprising
a) contacting a recombinant mammalian lymphoma cell according to claim 6 with a constant part of a monoclonal antibody, and
b) measuring expression of IL-2 from the recombinant mammalian lymphoma cell, wherein the strength of the expression of IL-2 is a measure of the strength of the interaction.

10. The method according to claim 9, wherein a simultaneous determination of the antigen specificity and the interaction takes place.

11. A method for identifying a compound that affects the interaction between constant parts of a monoclonal antibody and an Fc-receptor, comprising
a) contacting a recombinant mammalian lymphoma cell according to claim 6 with a constant part of a monoclonal antibody in the presence of a candidate compound,
b) measuring expression of IL-2 from the recombinant mammalian lymphoma cell, wherein the strength of the expression of IL-2 is a measure of the strength of the interaction, and
c) comparing the expression as measured in step b) with the IL-2 expression in the absence of the candidate compound.

12. The method according to claim 9, wherein the constant parts are present in soluble mAbs, in mAbs bound to plastics, in immune complexes or bound to target cells.

13. The method according to claim 9, wherein the constant parts are labeled and/or present in labeled mAbs.

14. The method according to claim 9, further comprising the generation of a binding profile of the constant part of mAbs of different subclasses for the individual Fc-receptors as expressed in the mammal.

15. The method according to claim 9, further comprising the modification of the constant part for increasing or decreasing the strength of the binding to an Fc-receptor.

16. A method for detecting Fc-gamma-receptor activating antibodies in a sample, comprising a method according to claim 9, wherein an expression of IL-2 is an indication of the presence of Fc-gamma-receptor activating antibodies in the sample.

17. The method according to claim 16, wherein the activating antibody is an autoimmune antibody.

18. The method according to claim 16, wherein the sample is analyzed in the context of an ontological disease, an infectious disease, an autoimmune disease, a disease of the musculo-skeletal system, an endocrine and/or metabolic functional disorder, a hematological disease, a respiratory disease, diseases of the CNS and/or an immunological disease.

19. The method according to claim 9, further comprising the modification of the candidate compound for increasing or decreasing the strength of the binding to an Fc-receptor.
